(19) United States
(12) Patent Application Publication
(10) Pub. No.: US 2017/0100277 A1
(43) Pub. Date: Apr. 13, 2017

(54) Method For Suppressing Snoring, And Smart Bed

(71) Applicant: Dongguan Maotian Precision Metal Products Co., Ltd., Dongguan City (CN)

(72) Inventor: Xiang KE, Dongguan City (CN)

(73) Assignee: Dongguan Maotian Precision Metal Products Co., Ltd., Dongguan City (CN)

(21) Appl. No.: 15/276,281

(22) Filed: Sep. 26, 2016

(30) Foreign Application Priority Data
Oct. 9, 2015 (CN) 201510649026.3

(51) Int. Cl. A61F 5/56; A47C 21/00; A47C 31/00; A47C 20/04

(52) U.S. Cl. CPC A61F 5/56; A47C 20/041; A47C 21/003; A47C 31/00

(57) ABSTRACT

The present disclosure discloses a method for suppressing snoring, based on which snoring is suppressed by adjusting an angle of a smart bed, specifically including following steps: acquiring sound in a room; judging whether the sound is snoring sound of the human body or not; keeping, if the sound is not snoring sound of a human body, a bed board at an existing angle, and returning to the step of acquiring the sound in the room; and adjusting the angle of the bed board in a pre-set way if the sound is snoring sound of a human body, and returning to the step of acquiring the sound in the room when the angle of the bed board is being adjusted or after the angle of the bed board is adjusted.

### CROSS REFERENCE TO RELATED APPLICATIONS

This application claims priority to Chinese Patent Application No. 201510649026.3, filed Oct. 9, 2015, the application of which is incorporated herein by reference, in its entirety.

### TECHNICAL FIELD

The present disclosure relates to the technical field of sleep snoring suppression, in particular to a method for suppressing snoring, and a smart bed.

### BACKGROUND

Snoring, also called making snore, is a prevalent sleep phenomenon, a lot of people believe that the snoring is common and does not matter in life, and some people even regard the snoring as the reflection of sound sleep. However, the snoring has an apparent adverse impact both on other people and on a snorer.

Firstly, the snoring influences the sleep of other people; the snorer is actually relatively deep and steady in sleep, but people sleeping in a same room with the snorer are very worried: when people who do not snore has not fallen asleep after lying in bed, the snorer has already fallen asleep and began to snore, the snoring sound would be noise influencing the sleep of people who do not snore, and the people who do not snore are dizzy and sleepy at a next day due to bad sleep and cannot focus on working and study.

Secondly, the snoring influences the health of the snorer; in the past, fewer people care about the danger of the snoring to the snorer; since the sleep becomes a research subject, the sleep quality of the snorer has been researched by researchers; and the research shows that: a long-term snorer or people with serious snoring always have a sleep apnea syndrome and have apnea in the whole sleep process, so that oxygen in the blood is reduced. That is to say, oxygen breathed by the snorer in the whole night is less than that breathed by a normal person, thereby influencing the memory after a long time. People test that a memory function of the snorer is actually worse than that of the normal person, which is not realized by the snorer. More seriously, sudden stoppage of breathing and heartbeat caused by the snoring may cause sudden death of people in sleep. Therefore, reducing or suppressing the snoring has a positive effect for other people and the snorer.

Based on the above situation, it is necessary to design a method for suppressing snoring so as to weaken and even eliminate the snoring phenomenon of the user.

### SUMMARY

An objective of the present disclosure is to provide a method for suppressing snoring, which judges whether there is snoring sound in a room or not and controls whether to adjust an angle of a bed board or not according to the snoring sound, so that the bed board is adjusted to the angle suitable for the sleep of a user, thereby suppressing the snoring of the user, and improving the sleep quality of the user.

An objective of the present disclosure is to provide a method for suppressing snoring, which effectively improves the precision for judging whether the sound is the snoring sound of the human body or not by recording personal snoring sound most approximate to the real snoring sound of a user.

An objective of the present disclosure is to provide a method for suppressing snoring, which firstly determines a loudness of sound in a room, and then compares the sound with conventional snoring sound and personal snoring sound, thereby effectively improving the precision for judging whether the sound is the snoring sound of the user, avoiding the fault adjustment for the bed board not to influence the sleep quality of the user.

An objective of the present disclosure is to provide a smart bed, which acquires sound in a room, adjusts an angle of a bed board according to the sound, and adjusts the bed board to an angle suitable for the sleep of a user, thereby suppressing the snoring of the user, and improving the sleep quality of the user.

In order to achieve the objectives, the present disclosure adopts a technical solution as follows:

In one aspect, a method for suppressing snoring is provided, based on which snoring is suppressed by adjusting an angle of a smart bed, specifically including following steps:

acquiring sound in a room;

judging whether the sound is snoring sound of a human body or not:

keeping, if the sound is not snoring sound of a human body, a bed board at an existing angle, and returning to the step of acquiring the sound in the room; and

adjusting the angle of the bed board in a pre-set way if the sound is snoring sound of a human body, and returning to the step of acquiring the sound in the room when the angle of the bed board is being adjusted or after the angle of the bed board is adjusted.

The medical research shows that any sound can be produced only by requiring the movement of various muscles in an oral cavity, a nasal cavity and a pharyngeal cavity, and the sound can be produced when airflow passes through various lacunas of different shapes formed by various muscles. When people speak, the sound is produced by impacting voids in the middle of vocal cords of the throat through the airflow, and muscles of lips, tongue, cheeks and jaws are cooperated to form cavities of various shapes, so that different initial consonants and simple or compound vowels are produced when the sound passes through the cavities to constitute a language. When people are in sleep, the muscles of the lips, the tongue, the cheeks and the jaws cannot be cooperated arbitrarily to form various cavities, but a large passage—throat (pharyngeal portion) is always reserved, so that if the passage is narrowed and becomes a gap, sound may be produced when the airflow passes through the gap, which is snoring. The present solution changes relative positions of the muscles of the lips, the tongue, the cheeks and the jaws when a user is in sleep by adjusting an inclination angle of the user, thereby preventing a throat passage from forming a relatively narrow gap, and further realizing a purpose of suppressing the snoring.

Specifically, if no sound can be acquired, it is determined that no snoring sound exists in the room, and a bed board is kept at an existing angle.

Specifically, an angle of the bed board refers to an angle between the bed board and a horizontal plane.

Specifically, one side of the bed board adjacent to the head of the human body is not lower than one side of the bed board adjacent to feet of the human body.

As a technical solution of a method for suppressing snoring, the step of judging whether the sound is the snoring sound of the human body specifically includes following steps:

pre-recording personal snoring sound of a user; and

comparing the acquired sound with the personal snoring sound, and judging whether the sound is the personal snoring sound or not.

Optionally, in order to meet requirements of a plurality of different users, a plurality of personal snoring sound data is pre-recorded.

Specifically, different users are different in snoring sound; the snoring sound of an individual user is relatively unique and is quite different from the normal snoring sound, so that the precision for judging whether the sound is the snoring sound of the human body or not can be effectively improved by recording the personal snoring sound most approximate to the actual snoring sound of the user. The recording of the personal snoring sound is completed by requiring manual assistance of a second person.

As a technical solution of a method for suppressing snoring, the step of judging whether the sound is the snoring sound of the human body specifically includes following steps:

reading conventional snoring sound of different people stored in a control system; and

comparing the acquired sound with the conventional snoring sound one by one, and judging whether the sound belongs to any conventional snoring sound or not.

Specifically, the conventional snoring sound is pre-stored in the control system when a smart bed is manufactured and does not need to be manually recorded by the user or the second person.

As a technical solution of a method for suppressing snoring, the step of judging whether the sound is the snoring sound of the human body is specifically as follows:

judging whether a loudness of the sound is greater than or equal to a pre-set snoring sound decibel value or not.

As a technical solution of a method for suppressing snoring, the step of judging whether the sound is the snoring sound of the human body is specifically as follows:

reading conventional snoring sound of different people stored in a control system, and pre-recording the personal snoring sound of a user;

judging whether a loudness of the sound is greater than or equal to a pre-set snoring sound decibel value or not:

returning to the step of acquiring the sound in the room if the loudness of the sound is not greater than or equal to the pre-set snoring sound decibel; and

comparing the acquired sound with the conventional snoring sound and the personal snoring sound one by one if the loudness of the sound is greater than or equal to the pre-set snoring sound decibel, and judging whether the sound belongs to any conventional snoring sound or personal snoring sound.

Specifically, firstly by determining the loudness of the sound in the room and then comparing the sound with the conventional snoring sound and the personal snoring sound, the precision for judging whether the sound is the snoring sound of the user can be effectively improved, thereby avoiding the fault adjustment for the bed board not to influence the sleep quality of the user.

As a technical solution of a method for suppressing snoring, the step of acquiring the sound in the room is specifically as follows:

capturing the sound once at a fixed duration interval.

Optionally, the fixed duration is any duration in a range of 10 s to 20 min.

Further, the fixed duration is 10 s, 25 s, 40 s, 1 min, 2 min, 3 min, 4 min, 5 min, 6 min, 7 min, 8 min, 9 min, 10 min, 11 min, 12 min, 13 min, 14 min, 15 min, 16 min, 17 min, 18 min, 19 min or 20 min.

As a technical solution of a method for suppressing snoring, the step of primarily adjusting the angle of the bed board in a pre-set way is specifically as follows:

judging whether the existing angle of the bed board is an upper limit angle or not:

adjusting, if the existing angle of the bed board is an upper limit angle, the angle of the bed board to a lower limit angle, and using the adjusted angle of the bed board as the existing angle; and

increasing, if the existing angle of the bed board is not an upper limit angle, the angle of the bed board by a constant angle, and using the adjusted angle of the bed board as the existing angle.

As a technical solution of a method for suppressing snoring, the step of primarily adjusting the angle of the bed board in a pre-set way is specifically as follows:

judging whether the existing angle of the bed board is an upper limit angle or not:

decreasing, if the existing angle of the bed board is an upper limit angle, the angle of the bed board by a constant angle, and using the adjusted angle of the bed board as the existing angle;

judging whether the existing angle of the bed board is a lower limit angle or not if the existing angle of the bed board is not an upper limit angle:

increasing, if the existing angle of the bed board is the lower limit angle, the angle of the bed board by a constant angle, and using the adjusted angle of the bed board as the existing angle;

judging whether the angle of the bed board is adjusted for the first time or not if the existing angle of the bed board is not the lower limit angle:

increasing, if the angle of the bed board is adjusted for the first time, the angle of the bed board by a constant angle, and using the adjusted angle of the bed board as the existing angle; and

increasing or decreasing, if the angle of the bed board is not adjusted for the first time, the angle of the bed board by a constant angle according to a previous adjustment direction for the angle of the bed board, and using the adjusted angle of the bed board as the existing angle.

Optionally, the upper limit angle is greater than 10 degrees and less than 15 degrees, and the lower limit angle is greater than 0 and less than 5 degrees.

Optionally, the adjustment constant angle at every time is greater than 0 and less than or equal to 5 degrees.

Further, the upper limit angle is 15 degrees, the lower limit angle is 0, and the adjustment constant angle at every time is one degree.

Further, the upper limit angle is 14 degrees, the lower limit angle is 0, and the adjustment constant angle at every time is 2 degrees.

Further, the upper limit angle is 13 degrees, the lower limit angle is one degree, and the adjustment constant angle at every time is 3 degrees.

As a technical solution of a method for suppressing snoring, after the step of acquiring the sound in the room, the method further includes following steps:

whether the adjustment for the angle of the bed board has already enabled the angle of the bed board to be cycled for two times between the upper limit angle and the lower limit angle is judged:

if the adjustment for the angle of the bed board has already enabled the angle of the bed board to be cycled for two times between the upper limit angle and the lower limit angle, acquisition of the sound in the room and adjustment for the angle of the bed board are ended; and

if the adjustment for the angle of the bed board has not enabled the angle of the bed board to be cycled for two times between the upper limit angle and the lower limit angle, the step of judging whether the sound is snoring sound of the human body or not is carried out.

Optionally, the above judgment condition may also be: whether the adjustment for the angle of the bed board has already enabled the angle of the bed board to be cycled for three times between the upper limit angle and the lower limit angle is judged, or whether the adjustment for the angle of the bed board has already enabled the angle of the bed board to be cycled for four times between the upper limit angle and the lower limit angle is judged.

Specifically, if the angle of the bed board is cycled for multiple times at different angles between the upper limit angle and the lower limit angle, and the snoring sound of the user still exists, it is considered that the adjustment for different angles of the bed board does not have an effect for suppressing the snoring sound of the user at the moment. Then acquisition of the sound in the room and adjustment for the angle of the bed board are ended, thereby avoiding the energy loss of the smart bed, and facilitating the energy conservation and environmental protection.

In another aspect, a smart bed is provided, which is used for realizing the method for suppressing snoring. The smart bed includes a base, a bed board disposed above the base, and a connecting support for movably connecting the base and the bed board; where the base or the bed board is provided with an audio input device for capturing sound and a control system, the audio input device is electrically connected with the control system, and the connecting support is controlled by the control system.

Optionally, the bed board includes a group of integrated boards moving synchronously.

Optionally, the bed board includes two groups of split-type single boards capable of separately moving, and the two groups of single boards are arranged in parallel along a width direction of the bed board.

As a technical solution of the smart bed, one end of the bed board adjacent to feet of the human body is provided with a limiting rod, and two ends of the limiting rod are fixed on the bed board through a duplex buckle. The limiting rod is configured to limit the slipping of a mattress or the human body at an inclination state of the bed board, so that the mattress or the human body can reliably stay on the bed board.

Optionally, the limiting rod is of an integrated structure, and two ends of the limiting rod are provided with a limiting slot corresponding to the duplex buckle.

As a technical solution of the smart bed, the duplex buckle includes a circular tubular buckle body, one end of the buckle body extends outwards along radial direction and is provided with a positioning boss, an outer side wall of one end of the buckle body away from the positioning boss is provided with an external connection boss, an inner side wall of middle portion of the buckle body is provided with an internal connection boss, and the external connection boss and the internal connection boss are opposite in snap-in directions.

Optionally, one end of the buckle body away from the positioning boss is provided with a V-shaped avoidance groove, and the V-shaped avoidance groove penetrates through the buckle body along the radial direction of the buckle body.

Specifically, the external connection boss is configured to fixedly install the duplex buckle onto the bed board, and the internal connection boss is configured to fixedly install the limiting rod in the duplex buckle, thereby realizing a fixed connection between the limiting rod and the bed board.

The present disclosure has the beneficial effects as follows:

(I) a method for suppressing snoring is provided, which judges whether there is snoring sound in a room and controls whether to adjust an angle of a bed board according to the snoring sound, so that the bed board is adjusted to an angle suitable for the sleep of a user, thereby suppressing the snoring of the user, and improving the sleep quality of the user.

(II) A snoring suppressing method is provided, which records the personal snoring sound most approximate to the actual snoring sound of the user, thereby effectively improving the precision for judging whether the sound is the snoring sound of the human body or not.

(III) A method for suppressing snoring is provided, which determines a loudness of the sound in the room, and then compares the sound with conventional snoring sound and the personal snoring sound, thereby effectively improving the precision for judging whether the sound is the snoring sound of the user, and avoiding the fault adjustment for the bed board not to influence the sleep quality of the user.

(IV) A smart bed is provided, which acquires sound in a room, and adjusts the angle of the bed board according to the sound, so that the bed board is adjusted to an angle suitable for the sleep of a user, thereby suppressing the snoring of the user, and improving the sleep quality of the user.

### BRIEF DESCRIPTION OF DRAWINGS

The present disclosure is further described below in detail according to drawings and embodiments.

FIG. 1 is a flow diagram illustrating a method for suppressing snoring provided in embodiments;

FIG. 2 is a flow diagram illustrating a method for suppressing snoring provided in embodiment I;

FIG. 3 is a flow diagram illustrating a method for suppressing snoring provided in embodiment II;

FIG. 4 is a flow diagram illustrating a method for suppressing snoring provided in embodiment III;

FIG. 5 is a schematic structural diagram illustrating a smart bed provided in embodiments;

FIG. 6 is a local schematic structural diagram illustrating a smart bed provided in embodiments;

FIG. 7 is a local enlarged view illustrating a position A as shown in FIG. 6;

FIG. 8 is a front view illustrating a duplex buckle provided in embodiments;

FIG. 9 is a top view illustrating a duplex buckle provided in embodiments;

FIG. 10 is a bottom view illustrating a duplex buckle provided in embodiments; and

FIG. 11 is a section view illustrating a B-B direction as shown in FIG. 9.

### REFERENCE NUMBERS

1. Base; 2. Connecting support; 3. Bed board; 4. Limiting rod; 41. Limiting slot; 5. Duplex buckle; 51. Buckle body; 52. Positioning boss; 53. External connection boss; 54. Internal connection boss; 55. V-shaped avoidance groove.

### DETAILED DESCRIPTION

The technical solution of the present disclosure is further described below with reference to drawings and embodiments.

### Embodiment I

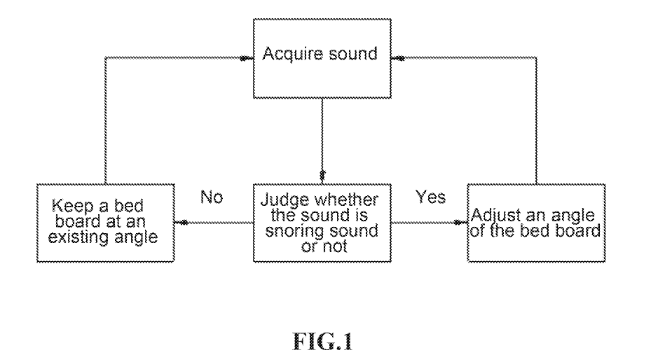

In one aspect, as shown in FIG. 1, provided is a method for suppressing snoring, which suppresses the snoring by adjusting an angle of a smart bed, and specifically includes following steps:

sound in a room is acquired;

whether the sound is snoring sound of a human body or not is judged:

if the sound is not snoring sound of a human body, a bed board is kept at an existing angle, and the method is returned to the step of acquiring the sound in the room; and

if the sound is snoring sound of a human body, the angle of the bed board is adjusted in a pre-set way, and the method is returned to the step of acquiring the sound in the room when the angle of the bed board is being adjusted or after the angle of the bed board is adjusted.

In present embodiment, after the step of adjusting the angle of the bed board in a pre-set way, the method is returned to the step of acquiring the sound in the room.

The medical research shows that any sound can be produced only by requiring the movement of various muscles in an oral cavity, a nasal cavity and a pharyngeal cavity, and the sound can be produced when airflow passes through various lacunas of different shapes formed by various muscles. When people speak, the sound is produced by impacting voids in the middle of vocal cords of the throat through the airflow, and muscles of lips, tongue, cheeks and jaws are cooperated to form cavities of various shapes, so that different initial consonants and simple or compound vowels are produced when the sound passes through the cavities to constitute a language. When people are in sleep, the muscles of the lips, the tongue, the cheeks and the jaws cannot be cooperated arbitrarily to form various cavities, but a large passage—throat (pharyngeal portion) is always reserved, so that if the passage is narrowed and becomes a gap, sound may be produced when the airflow passes through the gap, which is snoring. The present solution changes relative positions of the muscles of the lips, the tongue, the cheeks and the jaws when a user is in sleep by adjusting an inclination angle of the user, thereby preventing a throat passage from forming a relatively narrow gap, and further realizing a purpose of suppressing the snoring.

In present embodiment, if no sound can be acquired, it is determined that there is no snoring sound in a room, and a bed board is kept at an existing angle. An angle of the bed board refers to an angle between the bed board and a horizontal plane. One side of the bed board adjacent to the head of the human body is not lower than one side of the bed board adjacent to feet of the human body.

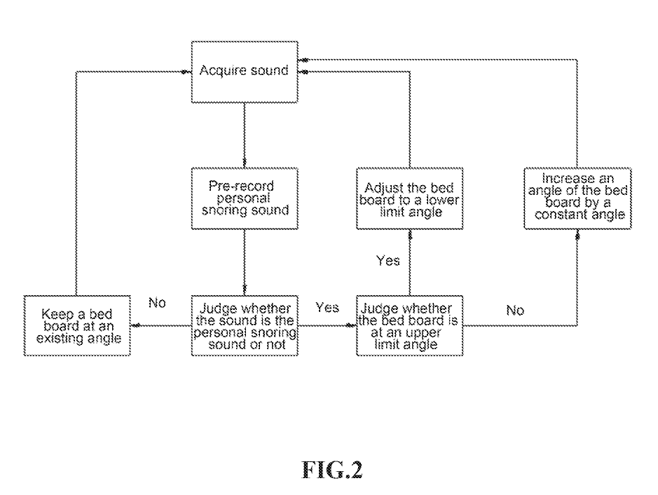

In present embodiment, as shown in FIG. 2, the step of judging whether the sound is the snoring sound of the human body specifically includes following steps:

personal snoring sound of a user is pre-recorded; and

the acquired sound is compared with the personal snoring sound, and whether the sound is the personal snoring sound or not is judged.

In order to meet requirements of a plurality of different users, a plurality of personal snoring sound data is pre-recorded. In present embodiment, the quantity of pre-recorded personal snoring sound data is two. Specifically, different users are different in snoring sound; and the snoring sound of an individual user is relatively unique and is quite different from the normal snoring sound, so that the precision for judging whether the sound is the snoring sound of the human body or not can be effectively improved by recording the personal snoring sound most approximate to the actual snoring sound of the user. The recording work of the personal snoring sound is completed by requiring the manual assistance of a second person.

In present embodiment, as shown in FIG. 2, the step of adjusting the angle of the bed board in a pre-set way is specifically as follows:

whether the existing angle of the bed board is an upper limit angle or not is judged:

if the existing angle of the bed board is an upper limit angle, the angle of the bed board is adjusted to a lower limit angle, and the adjusted angle of the bed board is used as the existing angle; and

if the existing angle of the bed board is not an upper limit angle, the angle of the bed board is increased by a constant angle, and the adjusted angle of the bed board is used as the existing angle.

The upper limit angle is greater than 10 degrees and less than 15 degrees, and the lower limit angle is greater than 0 and less than 5 degrees. The adjustment constant angle at every time is greater than 0 and less than or equal to 5 degrees. In present embodiment, the upper limit angle is 15 degrees, the lower limit angle is 0, and the adjustment constant angle at every time is one degree.

The step of acquiring the sound in the room is specifically as follows:

the sound is captured once at a fixed duration interval. The fixed duration is any duration in a range of 10 s to 20 min. In present embodiment, the fixed duration is 25 s.

In present embodiment, after the step of acquiring the sound in the room, the method further includes following steps:

whether the adjustment for the angle of the bed board has already enabled the angle of the bed board to be cycled for three times between the upper limit angle and the lower limit angle is judged:

if the adjustment for the angle of the bed board has already enabled the angle of the bed board to be cycled for three times between the upper limit angle and the lower limit angle, acquisition of the sound in the room and adjustment for the angle of the bed board are ended; and

if the adjustment for the angle of the bed board has not enabled the angle of the bed board to be cycled for three times between the upper limit angle and the lower limit angle, the step of judging whether the sound is snoring sound of the human body or not is carried out.

Specifically, if the angle of the bed board is cycled for multiple times at different angles between the upper limit angle and the lower limit angle, and the snoring sound of the user still exists, it is considered that the adjustment for different angles of the bed board does not have an effect for suppressing the snoring sound of the user at the moment. Then the acquisition of the sound in the room and adjustment for the angle of the bed board are ended, thereby avoiding the energy loss of the smart bed, and facilitating the energy conservation and environmental protection.

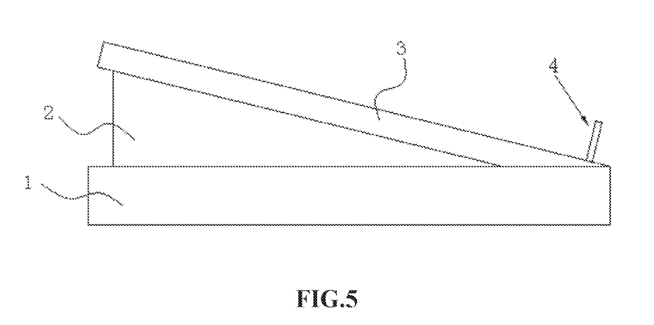

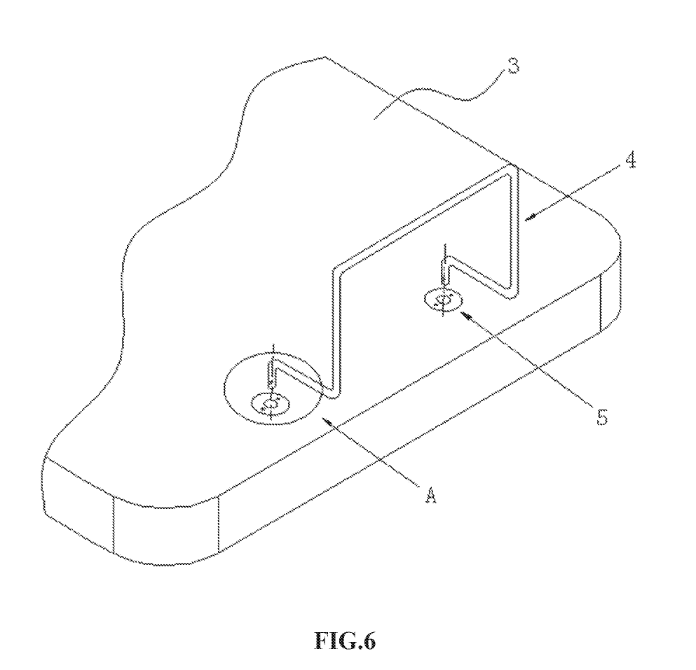

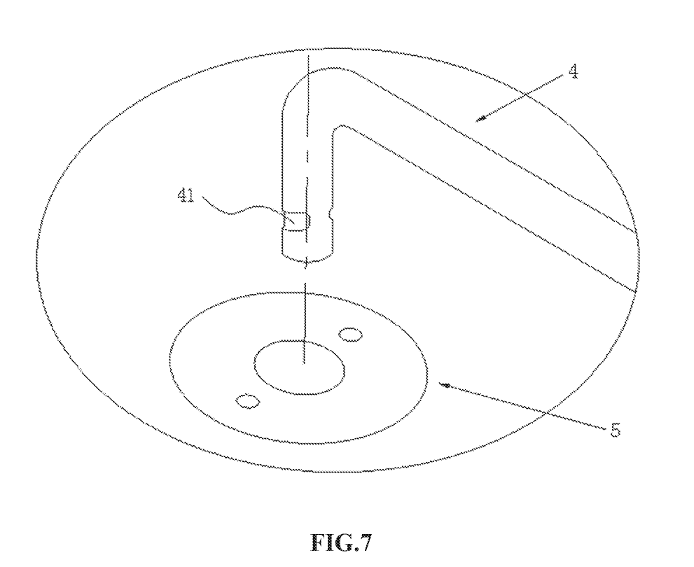

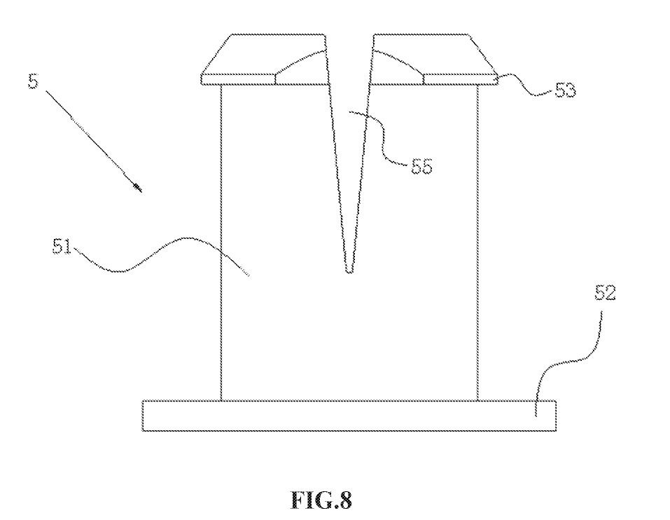

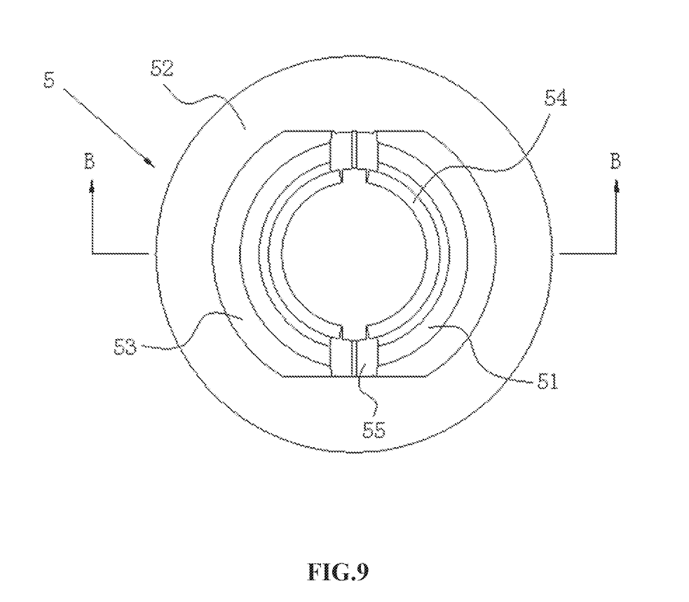

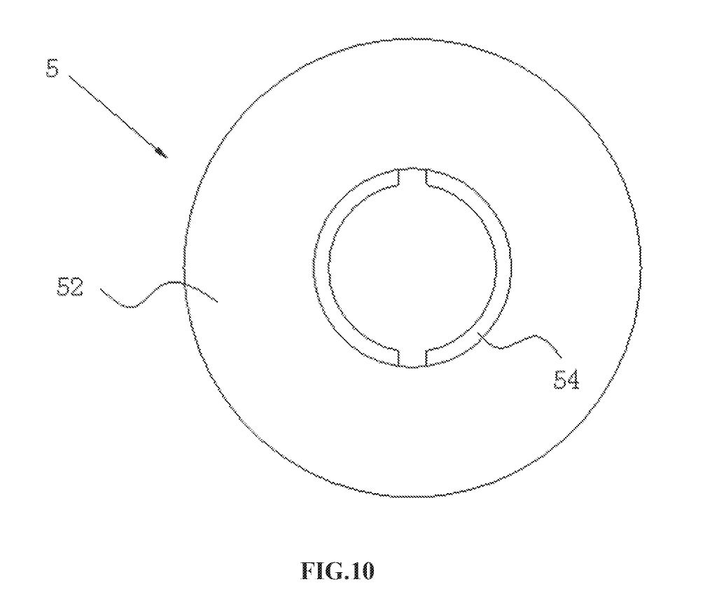

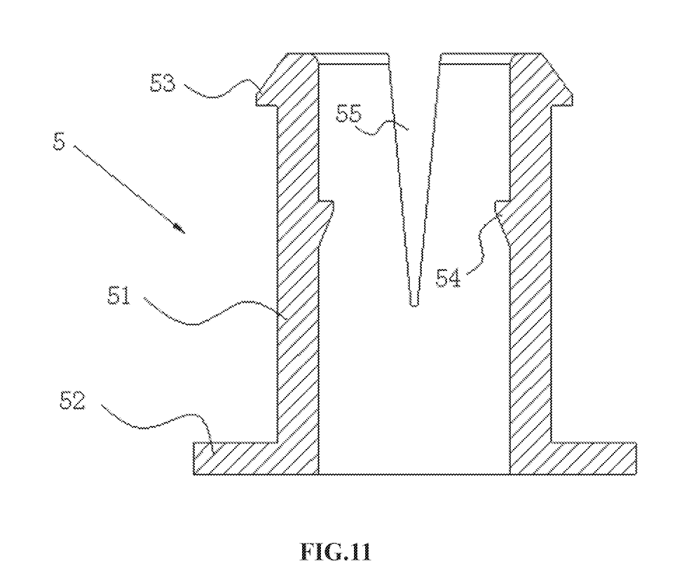

In another aspect, as shown in FIG. 5-11, a smart bed is provided, which is used for realizing the method for suppressing snoring. The smart bed includes a base 1, a bed board 3 disposed above the base 1 and a connecting support 2 for movably connecting the base 1 and the bed board 3, where the base 1 or the bed board 3 is provided with an audio input device for capturing sound and a control system, the audio input device is electrically connected with the control system, and the connecting support 2 is controlled by the control system. In present embodiment, the bed board 3 includes two groups of split-type single boards capable of separately moving, and the two groups of single boards are arranged in parallel along a width direction of the bed board 3. In other embodiments, the bed board 3 includes a group of integrated boards moving synchronously.

One end of the bed board 3 adjacent to feet of the human body is provided with a limiting rod 4, and two ends of the limiting rod 4 are fixed on the bed board 3 through a duplex buckle 5. The limiting rod 4 is configured to limit the slipping of a mattress or the human body at an inclination state of the bed board 3, so that the mattress or the human body can reliably stay on the bed board 3. The limiting rod 4 is of an integrated structure, and two ends of the limiting rod 4 are provided with a limiting slot 41 corresponding to the duplex buckle 5.

The duplex buckle 5 includes a circular tubular buckle body 51, one end of the buckle body 51 extends outwards along the radial direction to be provided with a positioning boss 52, the outer side wall of one end of the buckle body 51 away from the positioning boss 52 is provided with an external connection boss 53, the inner side wall of the middle portion of the buckle body 51 is provided with an internal connection boss 54, and the external connection boss 53 and the internal connection boss 54 are opposite in snap-in directions. One end of the buckle body 51 away from the positioning boss 52 is provided with a V-shaped avoidance groove 55, and the V-shaped avoidance groove 55 penetrates through the buckle body 51 along the radial direction of the buckle body 51. Specifically, the external connection boss 53 is configured to fixedly install the duplex buckle 5 onto the bed board 3, the internal connection boss 54 is configured to fixedly install the limiting rod 4 in the duplex buckle 5, thereby realizing a fixed connection between the limiting rod 4 and the bed board 3.

### Embodiment II

The present embodiment differs from the embodiment I in that:

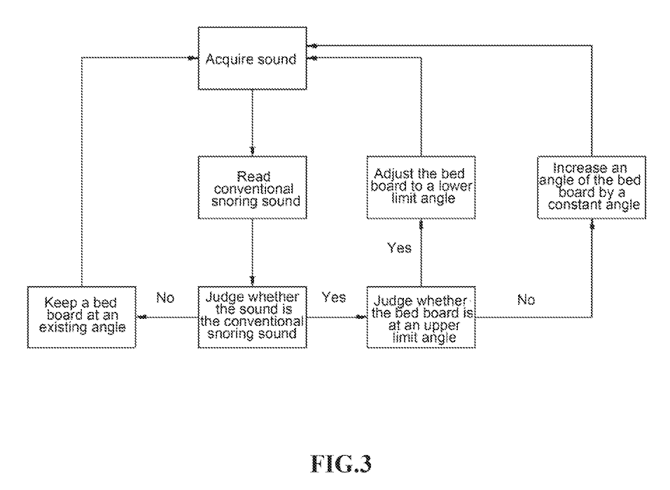

as shown in FIG. 3, the step of judging whether the sound is the snoring sound of the human body specifically includes following steps:

conventional snoring sound of different people stored in a control system is read; and

the acquired sound is compared with the conventional snoring sound one by one, and whether the sound belongs to any conventional snoring sound or not is judged.

In present embodiment, the conventional snoring sound is pre-stored in the control system when a smart bed is manufactured, and does not need to be manually recorded by a user or a second person.

In present embodiment, as shown in FIG. 3, the step of adjusting the angle of the bed board in a pre-set way is specifically as follows:

whether the existing angle of the bed board is an upper limit angle or not is judged:

if the existing angle of the bed board is an upper limit angle, the angle of the bed board is adjusted to a lower limit angle, and the adjusted angle of the bed board is used as the existing angle; and

if the existing angle of the bed board is not an upper limit angle, the angle of the bed board is increased by a constant angle, and the adjusted angle of the bed board is used as the existing angle.

The upper limit angle is greater than 10 degrees and less than 15 degrees, and the lower limit angle is greater than 0 and less than 5 degrees. The adjustment constant angle at every time is greater than 0 and less than or equal to 5 degrees. In present embodiment, the upper limit angle is 14 degrees, the lower limit angle is 0, and the adjustment constant angle at every time is 2 degrees.

The step of acquiring the sound in the room is specifically as follows:

the sound is captured once at a fixed duration interval. The fixed duration is any duration in a range of 10 s to 20 min. In present embodiment, the fixed duration is 10 s.

In present embodiment, after the step of acquiring the sound in the room, the method further includes following steps:

whether the adjustment for the angle of the bed board has already enabled the angle of the bed board to be cycled for two times between the upper limit angle and the lower limit angle:

if the adjustment for the angle of the bed board has already enabled the angle of the bed board to be cycled for two times between the upper limit angle and the lower limit angle, acquisition of the sound in the room and adjustment for the angle of the bed board are ended; and

if the adjustment for the angle of the bed board has not enabled the angle of the bed board to be cycled for two times between the upper limit angle and the lower limit angle, the step of judging whether the sound is snoring sound of the human body or not is carried out.

### Embodiment III

The present embodiment differs from the embodiment I in that:

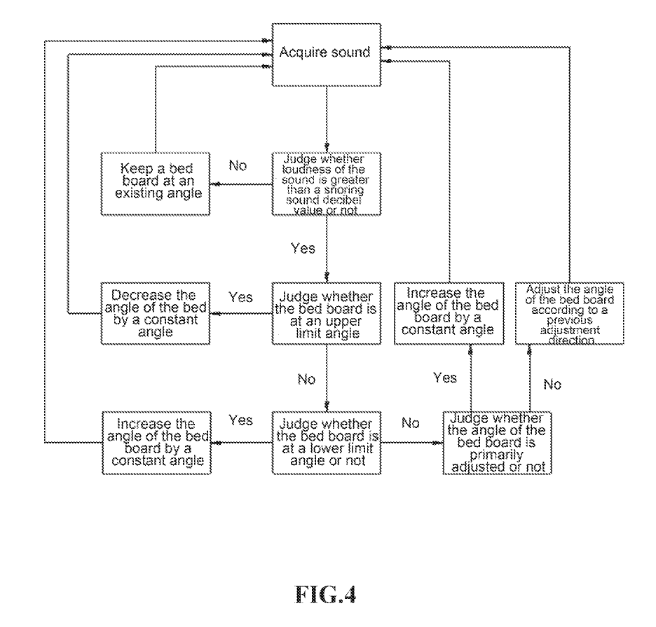

as shown in FIG. 4, the step of judging whether the sound is the snoring sound of the human body is specifically as follows:

whether a loudness of the sound is greater than or equal to a pre-set snoring sound decibel value or not is judged.

In present embodiment, as shown in FIG. 4, the step of adjusting the angle of the bed board in a pre-set way is specifically as follows:

whether the existing angle of the bed board is an upper limit angle or not is judged:

if the existing angle of the bed board is an upper limit angle, the angle of the bed board is decreased by a constant angle, and the adjusted angle of the bed board is used as the existing angle;

if the existing angle of the bed board is not an upper limit angle, whether the existing angle of the bed board is the lower limit angle or not is judged:

if the existing angle of the bed board is the lower limit angle, the angle of the bed board is increased by a constant angle, and the adjusted angle of the bed board is used as the existing angle;

if the existing angle of the bed board is not the lower limit angle, whether the angle of the bed board is adjusted for the first time or not is judged:

if the angle of the bed board is adjusted for the first time, the angle of the bed board is increased by a constant angle, and the adjusted angle of the bed board is used as the existing angle;

if the angle of the bed board is not adjusted for the first time, the angle of the bed board is increased or decreased by a constant angle according to a previous adjustment direction for the angle of the bed board, and the adjusted angle of the bed board is used as the existing angle.

The upper limit angle is greater than 10 degrees and less than 15 degrees, and the lower limit angle is greater than 0 and less than 5 degrees. The adjustment constant angle at every time is greater than 0 and less than or equal to 5 degrees. In present embodiment, the upper limit angle is 13 degrees, the lower limit angle is one degree, and the adjustment constant angle at every time is 3 degrees.

The step of acquiring the sound in the room is specifically as follows:

the sound is captured once at a fixed duration interval. The fixed duration is any duration in a range of 10 s to 20 min. In present embodiment, the fixed duration is 10 min.

In present embodiment, after the step of acquiring the sound in the room, the method further includes following steps:

whether the adjustment for the angle of the bed board has already enabled the angle of the bed board to be cycled for four times between the upper limit angle and the lower limit angle:

if the adjustment for the angle of the bed board has already enabled the angle of the bed board to be cycled for four times between the upper limit angle and the lower limit angle, acquisition of the sound in the room and adjustment for the angle of the bed board are ended; and

if the adjustment for the angle of the bed board has not enabled the angle of the bed board to be cycled for four times between the upper limit angle and the lower limit angle, the step of judging whether the sound is snoring sound of the human body or not is carried out.

### Embodiment IV

The present embodiment differs from the embodiment I in that:

the step of judging whether the sound is the snoring sound of the human body is specifically as follows:

conventional snoring sound of different people stored in a control system is read, and the personal snoring sound of the user is pre-recorded;

whether a loudness of the sound is greater than or equal to a pre-set snoring sound decibel value or not is judged:

if the loudness of the sound is not greater than or equal to a pre-set snoring sound decibel value, the method is returned to the step of acquiring the sound in the room; and

if the loudness of the sound is greater than or equal to a pre-set snoring sound decibel value, the acquired sound is compared with the conventional snoring sound and the personal snoring sound one by one, and whether the sound belongs to any conventional snoring sound or the personal snoring sound is judged.

Specifically, firstly by determining the loudness of the sound in the room and then comparing the sound with the conventional snoring sound and the personal snoring sound, the precision for judging whether the sound is the snoring sound of the user can be effectively improved, thereby avoiding the fault adjustment for the bed board not to influence the sleep quality of the user.

In present embodiment, the step of adjusting the angle of the bed board in a pre-set way is specifically as follows:

whether the existing angle of the bed board is an upper limit angle or not is judged:

if the existing angle of the bed board is an upper limit angle, the angle of the bed board is decreased by a constant angle, and the adjusted angle of the bed board is used as the existing angle;

if the existing angle of the bed board is not an upper limit angle, whether the existing angle of the bed board is the lower limit angle or not is judged:

if the existing angle of the bed board is the lower limit angle, the angle of the bed board is increased by a constant angle, and the adjusted angle of the bed board is used as the existing angle;

if the existing angle of the bed board is not the lower limit angle, whether the angle of the bed board is adjusted for the first time or not is judged:

if the angle of the bed board is adjusted for the first time, the angle of the bed board is increased by a constant angle, and the adjusted angle of the bed board is used as the existing angle;

if the angle of the bed board is not adjusted for the first time, the angle of the bed board is increased or decreased by a constant angle according to a previous adjustment direction for the angle of the bed board, and the adjusted angle of the bed board is used as the existing angle.

The upper limit angle is greater than 10 degrees and less than 15 degrees, and the lower limit angle is greater than 0 and less than 5 degrees. The adjustment constant angle at every time is greater than 0 and less than or equal to 5 degrees. In present embodiment, the upper limit angle is 15 degrees, the lower limit angle is 0, and the adjustment constant angle at every time is 5 degrees.

The step of acquiring the sound in the room is specifically as follows:

the sound is captured once at a fixed duration interval. The fixed duration is any duration in a range of 10 s to 20 min. In present embodiment, the fixed duration is 20 min.

In present embodiment, after the step of acquiring the sound in the room, the method further includes following steps:

whether the adjustment for the angle of the bed board has already enabled the angle of the bed board to be cycled for three times between the upper limit angle and the lower limit angle is judged:

if the adjustment for the angle of the bed board has already enabled the angle of the bed board to be cycled for three times between the upper limit angle and the lower limit angle, acquisition of the sound in the room and adjustment for the angle of the bed board are ended; and

if the adjustment for the angle of the bed board has not enabled the angle of the bed board to be cycled for three times between the upper limit angle and the lower limit angle, the step of judging whether the sound is snoring sound of the human body or not is carried out.

It should be stated that the above embodiments are only embodiments and technical principles used in the present disclosure; and any changes or replacements, which are easily conceived by those skilled in the art, in the disclosure of the present disclosure should fall within the protection scope of the present disclosure.

1. A method for suppressing snoring, based on which snoring is suppressed by adjusting an angle of a smart bed, comprising:
acquiring sound in a room;
judging whether the sound is snoring sound of a human body or not:
keeping, if the sound is not snoring sound of a human body, a bed board at an existing angle, and returning to the step of acquiring the sound in the room; and
adjusting the angle of the bed board in a pre-set way if the sound is snoring sound of a human body, and returning to the step of acquiring the sound in the room when the angle of the bed board is being adjusted or after the angle of the bed board is adjusted.

2. The method for suppressing snoring according to claim 1, wherein a step of judging whether the sound is the snoring sound of the human body specifically comprises following steps:
pre-recording personal snoring sound of a user; and
comparing the acquired sound with the personal snoring sound, and judging whether the sound is the personal snoring sound or not.

3. The method for suppressing snoring according to claim 1, wherein a step of judging whether the sound is the snoring sound of the human body specifically comprises following steps:
reading conventional snoring sound of different people stored in a control system; and
comparing the acquired sound with the conventional snoring sound one by one, and judging whether the sound belongs to any conventional snoring sound or not.

4. The method for suppressing snoring according to claim 1, wherein a step of judging whether the sound is the snoring sound of the human body is specifically as follows:
judging whether a loudness of the sound is greater than or equal to a pre-set snoring sound decibel value or not.

5. The method for suppressing snoring according to claim 1, wherein a step of judging whether the sound is the snoring sound of the human body is specifically as follows:
reading conventional snoring sound of different people stored in a control system, and pre-recording personal snoring sound of a user;
judging whether a loudness of the sound is greater than or equal to a pre-set snoring sound decibel value or not:
returning to the step of acquiring the sound in the room if the loudness of the sound is not greater than or equal to the pre-set snoring sound decibel; and
comparing the acquired sound with the conventional snoring sound and the personal snoring sound one by one if the loudness of the sound is greater than or equal to the pre-set snoring sound decibel, and judging whether the sound belongs to any conventional snoring sound or personal snoring sound.

6. The method for suppressing snoring according to claim 1, wherein a step of acquiring the sound in the room is specifically as follows:
capturing the sound once at a fixed duration interval.

7. The method for suppressing snoring according to claim 1, wherein the step of adjusting the angle of the bed board in a pre-set way is specifically as follows:
judging whether the existing angle of the bed board is an upper limit angle or not:
adjusting, if the existing angle of the bed board is an upper limit angle, the angle of the bed board to a lower limit angle, and using the adjusted angle of the bed board as the existing angle; and
increasing, if the existing angle of the bed board is not an upper limit angle, the angle of the bed board by a constant angle, and using the adjusted angle of the bed board as the existing angle.

8. The method for suppressing snoring according to claim 1, wherein the step of adjusting the angle of the bed board in a pre-set way is specifically as follows:
judging whether the existing angle of the bed board is an upper limit angle or not:
decreasing, if the existing angle of the bed board is an upper limit angle, the angle of the bed board by a constant angle, and using the adjusted angle of the bed board as the existing angle;
judging whether the existing angle of the bed board is a lower limit angle or not if the existing angle of the bed board is not an upper limit angle:
increasing, if the existing angle of the bed board is the lower limit angle, the angle of the bed board by a constant angle, and using the adjusted angle of the bed board as the existing angle;
judging whether the angle of the bed board is adjusted for the first time or not if the existing angle of the bed board is not the lower limit angle:
increasing, if the angle of the bed board is adjusted for the first time, the angle of the bed board by a constant angle, and using the adjusted angle of the bed board as the existing angle; and
increasing or decreasing, if the angle of the bed board is not adjusted for the first time, the angle of the bed board by a constant angle according to a previous adjustment direction for the angle of the bed board, and using the adjusted angle of the bed board as the existing angle.

9. A smart bed, for realizing the method for suppressing snoring of claim 1, comprising: a base, a bed board disposed above the base, and a connecting support for movably connecting the base and the bed board; wherein the base or the bed board is provided with an audio input device for capturing sound and a control system, the audio input device is electrically connected with the control system, and the connecting support is controlled by the control system.

10. The smart bed according to claim 9, wherein one end of the bed board adjacent to feet of the human body is provided with a limiting rod, and two ends of the limiting rod are fixed on the bed board through a duplex buckle; the duplex buckle comprises a circular tubular buckle body; one end of the buckle body extends outwards along radial direction and is provided with a positioning boss; an outer side wall of one end of the buckle body away from the positioning boss is provided with an external connection boss; an inner side wall of middle portion of the buckle body is provided with an internal connection boss; and the external connection boss and the internal connection boss are opposite in snap-in directions.

11. The method for suppressing snoring according to claim 2, wherein a step of acquiring the sound in the room is specifically as follows:
capturing the sound once at a fixed duration interval.

12. The method for suppressing snoring according to claim 3, wherein a step of acquiring the sound in the room is specifically as follows:
capturing the sound once at a fixed duration interval.

13. The method for suppressing snoring according to claim 4, wherein a step of acquiring the sound in the room is specifically as follows:
capturing the sound once at a fixed duration interval.

14. The method for suppressing snoring according to claim 5, wherein a step of acquiring the sound in the room is specifically as follows:
capturing the sound once at a fixed duration interval.

15. The method for suppressing snoring according to claim 2, wherein the step of adjusting the angle of the bed board in a pre-set way is specifically as follows:
judging whether the existing angle of the bed board is an upper limit angle or not:
adjusting, if the existing angle of the bed board is an upper limit angle, the angle of the bed board to a lower limit angle, and using the adjusted angle of the bed board as the existing angle; and
increasing, if the existing angle of the bed board is not an upper limit angle, the angle of the bed board by a constant angle, and using the adjusted angle of the bed board as the existing angle.

16. The method for suppressing snoring according to claim 3, wherein the step of adjusting the angle of the bed board in a pre-set way is specifically as follows:
judging whether the existing angle of the bed board is an upper limit angle or not:
adjusting, if the existing angle of the bed board is an upper limit angle, the angle of the bed board to a lower limit angle, and using the adjusted angle of the bed board as the existing angle; and
increasing, if the existing angle of the bed board is not an upper limit angle, the angle of the bed board by a constant angle, and using the adjusted angle of the bed board as the existing angle.

17. The method for suppressing snoring according to claim 4, wherein the step of adjusting the angle of the bed board in a pre-set way is specifically as follows:
judging whether the existing angle of the bed board is an upper limit angle or not:
adjusting, if the existing angle of the bed board is an upper limit angle, the angle of the bed board to a lower limit angle, and using the adjusted angle of the bed board as the existing angle; and
increasing, if the existing angle of the bed board is not an upper limit angle, the angle of the bed board by a constant angle, and using the adjusted angle of the bed board as the existing angle.

18. The method for suppressing snoring according to claim 5, wherein the step of adjusting the angle of the bed board in a pre-set way is specifically as follows:
judging whether the existing angle of the bed board is an upper limit angle or not:
adjusting, if the existing angle of the bed board is an upper limit angle, the angle of the bed board to a lower limit angle, and using the adjusted angle of the bed board as the existing angle; and
increasing, if the existing angle of the bed board is not an upper limit angle, the angle of the bed board by a constant angle, and using the adjusted angle of the bed board as the existing angle.

19. The method for suppressing snoring according to claim 2, wherein the step of adjusting the angle of the bed board in a pre-set way is specifically as follows:
judging whether the existing angle of the bed board is an upper limit angle or not:
decreasing, if the existing angle of the bed board is an upper limit angle, the angle of the bed board by a constant angle, and using the adjusted angle of the bed board as the existing angle;
judging whether the existing angle of the bed board is a lower limit angle or not if the existing angle of the bed board is not an upper limit angle:
increasing, if the existing angle of the bed board is the lower limit angle, the angle of the bed board by a constant angle, and using the adjusted angle of the bed board as the existing angle;
judging whether the angle of the bed board is adjusted for the first time or not if the existing angle of the bed board is not the lower limit angle:
increasing, if the angle of the bed board is adjusted for the first time, the angle of the bed board by a constant angle, and using the adjusted angle of the bed board as the existing angle; and
increasing or decreasing, if the angle of the bed board is not adjusted for the first time, the angle of the bed board by a constant angle according to a previous adjustment direction for the angle of the bed board, and using the adjusted angle of the bed board as the existing angle.

20. The method for suppressing snoring according to claim 3, wherein the step of adjusting the angle of the bed board in a pre-set way is specifically as follows:
judging whether the existing angle of the bed board is an upper limit angle or not:
decreasing, if the existing angle of the bed board is an upper limit angle, the angle of the bed board by a constant angle, and using the adjusted angle of the bed board as the existing angle;
judging whether the existing angle of the bed board is a lower limit angle or not if the existing angle of the bed board is not an upper limit angle:
increasing, if the existing angle of the bed board is the lower limit angle, the angle of the bed board by a constant angle, and using the adjusted angle of the bed board as the existing angle;
judging whether the angle of the bed board is adjusted for the first time or not if the existing angle of the bed board is not the lower limit angle:
increasing, if the angle of the bed board is adjusted for the first time, the angle of the bed board by a constant angle, and using the adjusted angle of the bed board as the existing angle; and
increasing or decreasing, if the angle of the bed board is not adjusted for the first time, the angle of the bed board by a constant angle according to a previous adjustment direction for the angle of the bed board, and using the adjusted angle of the bed board as the existing angle.
